(19) United States
(12) Patent Application Publication
(10) Pub. No.: US 2012/0310129 A1
(43) Pub. Date: Dec. 6, 2012

(54) Focal Compression Therapeutic Device

(76) Inventor: Donald E. Johnson, San Diego, CA (US)

(21) Appl. No.: 13/153,418

(22) Filed: Jun. 4, 2011

(51) Int. Cl. A61F 5/00

(52) U.S. Cl. 602/20

(57) ABSTRACT

A device for exerting therapeutic pressure on a user's elbow, the device comprising a sleeve sized for snugly encircling an arm in the region of the elbow, a pocket disposed on a length of a surface of the sleeve, sized for accommodating a pressure wad, which under control of the user can be registered and exert pressure on an anatomical point of an elbow.

### BACKGROUND

1. Field of the Invention

This invention generally relates to a therapeutic device, in particular to a sleeve or wrap having a focal pressure wad for applying a focal pressure to the elbow region to relieve stress conditions of the myofascial structure, for example “tennis elbow”.

2. Description of the Related Art

Tennis elbow is a condition where the outer part of the elbow becomes sore and tender. It is commonly associated with playing tennis and other racquet sports. Almost anyone, however, is subject to the injury.

The condition is also known as lateral epicondylitis (“inflammation of the outside elbow bone”). Other descriptions for tennis elbow are lateral epicondylosis, lateral epicondylalgia, or simply lateral elbow pain. The muscular structures as well as their fascial and tendon attachments are frequently the site of pain resulting from muscle tightness, restricted motion and/or inflammation.

The symptoms of tennis elbow are typically pain on the outer part of the elbow (lateral epicondyle), point tenderness over the lateral epicondyle—a prominent part of the bone on the outside of the elbow, pain caused by gripping and movements of the wrist, especially wrist extension and lifting movements; activities that use the muscles that extend the wrist (e.g. pouring a pitcher or gallon of milk, lifting with the palm down) are characteristically painful; and morning stiffness.

### Treatment

In general there is little evidence for effective measures of intervention. Non-specific palliative treatments include: physical therapy, non-steroidal anti-inflammatory drugs (NSAIDs): ibuprofen, naproxen or aspirin; heat or ice.

Other common modes for prevention, treatment and avoidance of recurrence are largely speculative. (Boyer M I, Hastings H (1999). “Lateral tennis elbow: “Is there any science out there?”. Journal of Shoulder and Elbow Surgery 8 (5): 481-91.) These modes include stretches and progressive strengthening exercises to prevent re-irritation of the tendon; progressive strengthening involving use of weights or elastic bands to promote pain-free grip strength and forearm strength. Racquet sport players are commonly advised to strengthen their shoulder rotator cuff, scapulothoracic and abdominal muscles by physiotherapists to help reduce any overcompensation in the wrist extensors during gross shoulder and arm movements. Soft tissue release or simply massage can help reduce the muscular tightness and reduce the tension on the tendons; and strapping of the forearm can help realign the muscle fibers and redistribute the load. Therapeutic effects are also sought through use of a racket designed to dampen the effect of ball striking; and a variety of tennis elbow braces known as counter-force brace or “tennis elbow strap” to reduce strain at the elbow epicondyle, to limit pain provocation and to protect against further damage (e.g. U.S. Pat. No. 3,970,081). There is little evidence to support the value of these interventions for prevention, treatment, or avoidance of recurrence of lateral epicondylosis.

The concept of counterforce bracing has been implemented by devices which encircle the extremity, creating pressure around and against the involved myofascial structures. The theoretical effect of counterforce bracing has been described as disseminating force applied to a muscle over a wider area or reducing force generated by the muscle below a level that would be less likely to induce irritation at its point of attachment to the bone.

Currently available bracing devices used for this purpose generally consist of simple elastic or non-elastic bands which encircle the extremity and apply force around the limb in a uniform manner. In addition, a tennis elbow brace is known which utilizes a pre-inflated air pillow or soft gel pad held against the involved muscle by an encircling wrap.

However, such conventional bracing devices suffer various drawbacks. The simple elastic or non-elastic band, for example, is deficient since the pressure is uniformly applied and not sufficiently concentrated at the affected area. On the other hand, the above-mentioned pre-inflated air pillow or soft gel pad configuration covers a somewhat limited surface area and can tend to move around along the surface of the forearm during use since it is not particularly adapted to engage the contour of the underlying limb and musculature.

Accordingly, there is a need for a device for effectively reducing pain in the elbow associated with the injury of tennis elbow.

### SUMMARY

It is an object of the invention to provide a device which may be worn about a user's elbow for alleviating elbow pain.

It is another object of the invention to provide a device which may be easily and quickly put onto the user's arm and worn; and easily and quickly removed when the user desires.

It is still another object of the invention to provide a device which is adjustable to a position on the user's arm according to the size of the user's arm and elbow and location of pain in the user's elbow.

Object of the present invention is to provide a tennis elbow compression device the position of which on the arm and elbow region can be easily adjusted by the user who is empirically determining which position of focal compression provides optimum relief, thereby adjusting the position of the device to provide that focal pressure and relief.

It is another object of the present invention to provide an improved therapeutic device for treating tennis elbow specifically intended for exerting focal pressure on a desired location on the body, capable of holding a pressure wad adapted in shape for focally applying pressure in place at a desired location on the body, without substantially restricting body movement.

Without limitation in light of said objectives, the device of the invention, comprises a sleeve sized for snugly encircling an arm in the region of the elbow. A pocket is disposed on a length of a surface of the sleeve. In certain embodiments, the pocket is positioned on an inner surface of the sleeve. The pocket is sized for receiving a pressure wad, a portion of which comprises a rounded surface. The device comprises a configuration in which the user adjusts either the position of the sleeve and/or the position of the pressure wad in the pocket to achieve registration of a rounded surface of the pressure wad exerting pressure on an anatomical point on the elbow.

Embodiments of the sleeve range in length from about four inches to about ten inches. The sleeve is formed from one or more materials selected from the group consisting of one-way stretch material, two-way stretch material, and non-stretchable material. In certain embodiments, an inner surface of the device comprises a soft, pliable material compatible with human skin.

Certain embodiments of the device comprise a wrap having an inner surface and an outer surface, free ends, and sized to enclose at least a portion of the user's arm above and below the elbow. Releasable securements are affixed on free ends of the wrap to permit the closure of the wrap around at least portions of the user's arm above and below the elbow; A pocket formed on the inner surface of the wrap embodiment of the device contains a pressure wad having a surface a portion of which is rounded. In use, the user positions the wrap around the elbow region, registers the securements into engagement with each other thereby forming a sleeve secured around the elbow region. The pressure wad is registered and exerts pressure against a point on the user's elbow.

The nature and construction of the present invention, along with the foregoing and other objects and advantages thereof, will become more readily apparent from the following description of disclosed embodiments shown and described with respect to the drawings.

### DRAWINGS

FIG. 1 shows the device of the present invention positioned on the wearer's arm in the region of the elbow.

FIG. 2 is a sectional view through 2-2 of FIG. 1.

FIG. 3 is a perspective view of the device which shows a rounded pressure wad and one end of a pocket on the inner surface of the sleeve into which the pressure wad is positioned.

FIG. 4 is a perspective view of the lower end of the device which shows in ghost the pressure wad positioned in a pocket

FIG. 5 is a perspective view of the upper end of the device which shows the deformation of the outer surface of the sleeve due to the presence of the pressure wad positioned in a pocket.

FIG. 6 is a cross sectional view along line 6-6 of FIG. 5.

FIG. 7 is a perspective view of the device which shows in ghost an elongate pocket and a pressure wad positioned in the pocket.

### DETAILED DESCRIPTION

The foregoing and other objects are accomplished according to the present invention, which includes an elongated flexible sleeve 1 of soft, stretchable having sufficient length and width which accommodates or encircles the girth of the user's limb 5, and having one or more pressure wads 10 positioned in one or more pockets 15 on the inner surface of the sleeve.

The soft fabric of the sleeve is stretchable at least in a circumferential direction. By way of example, but not limitation, a material suitable for making the sleeve is neoprene.

A pocket 20 formed on the inner surface 25 of said sleeve has an opening 30 on one or both ends in which fits a pressure wad 10. Positioned within the pocket 20 and juxtaposed by the user's placement of the device on his or her arm, the pressure wad 10 exerts focal compression against the elbow. The position of the pocket 20 and the position of the pressure wad 10 within the pocket can be changed as per the requirement of the wearer.

In one respect, the device includes main components: a sleeve 25, a pocket 20 mounted on the inner surface 25 of the sleeve 30, and a pressure wad 10 positioned or mounted within the pocket 20.

In certain embodiments of the device, the pocket 20, which may be made from the same or different soft, flexible material as the remainder of the sleeve, is formed on or secured to the inner surface 25 of the sleeve 30 by stitching 36, or by adhesive coating of a wide range of materials including: felt, foam, fabric, rubber, and film; laminating and laminating materials by which direct coating pressure sensitive and heat-activated adhesives are applied to a variety of soft or flexible materials. One of skill in the art has available well known companies (e.g. Associated Industries, Inc., Wichita, Kans.) which provide a variety of technologies and materials for securing soft materials to each other.

When desired, the device 1 is worn when the user slips his or her forearm through the elastic sleeve 30, adjusting the position of the device to achieve registration of the pressure wad 10 on the desired point of the elbow, wherein the radially inward force achieved by the sleeve's elasticity against the user's arm actuates focal pressure of the pressure wad against the desired point of the elbow. The elasticity of the sleeve 30 is itself sufficient to hold the device in place on the user's arm.

The present invention has particular utility in the field of treating elbow joint pain, commonly observed in tennis players but a condition incident to many other activities of the user's arm and concomitant pathologies.

### Sleeve

This invention relates generally to tubular devices, commonly sleeves 30, applicable to the elbow of a user.

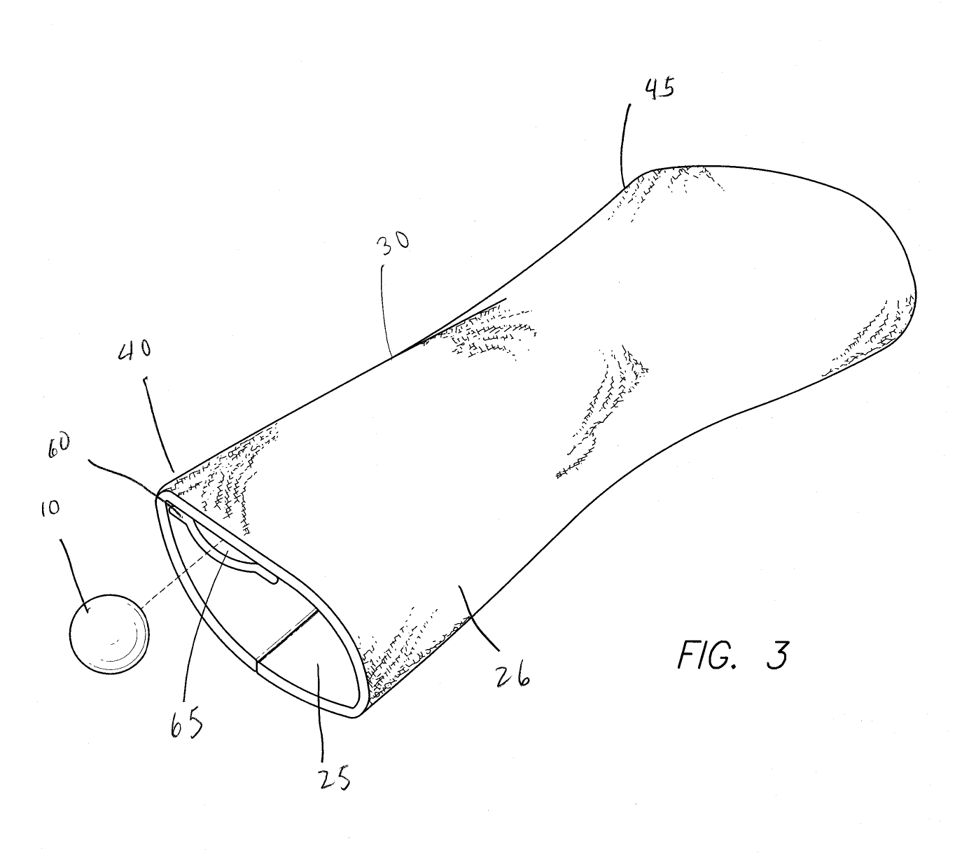

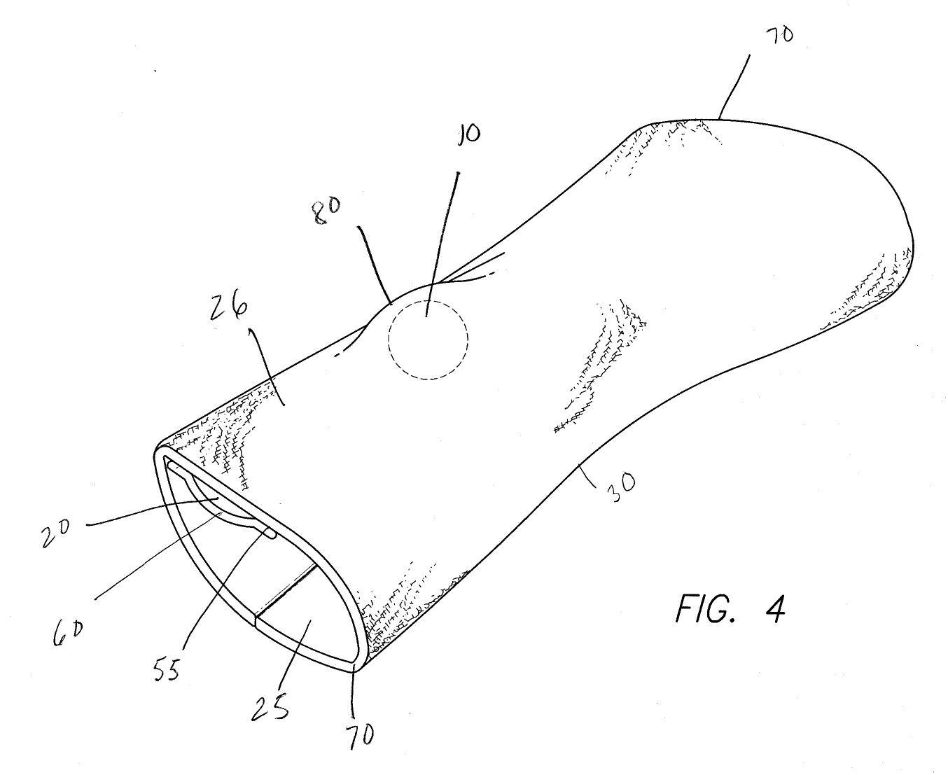

The sleeve has opposite ends 40, 45. When worn or applied to the user's arm, the sleeve fits closely or snugly to the user's elbow. The taper of the sleeve from its upper to its lower end as indicated by the taper seen in FIGS. 3, 4, 5 and 7 conforms to the user's arm taper above and below the elbow area.

The sleeve 30 may include one or more layers of elastomeric material 50 (e.g. neoprene), said layers each having a thickness between approximately ⅛ inch and ¼ inch.

The inner surface 25 of the sleeve 30 comprises one or more pockets 20, which may run the length of the device, i.e. from end to end; or the length of a pocket may terminate at a position spaced from one or both ends.

The sleeve 30 has a longitudinal dimension. The longitudinal dimension is herein defined as the dimension which is fitted on a user's limb, in particular, an elbow.

In one embodiment, the elongated flexible sleeve of the present invention is substantially nonelastic in its longitudinal dimension, yet has sufficient circumferential (lateral) elasticity to permit substantial stretching to accommodate the girth of the user's limb. The circumferential or lateral resiliency of the device permits the device to be firmly held in place about the user's limb, in particular, on a flexible elbow joint, or even, in certain embodiments, a knee joint, while permitting circumferential resiliency to accommodate body movements. At the same time, the relatively inelastic length of the sleeve prevents the sleeve from undergoing longitudinal elongation during body movement, so that the sleeve tends to remain affixed and exerting circumferential pressure in position on the body.

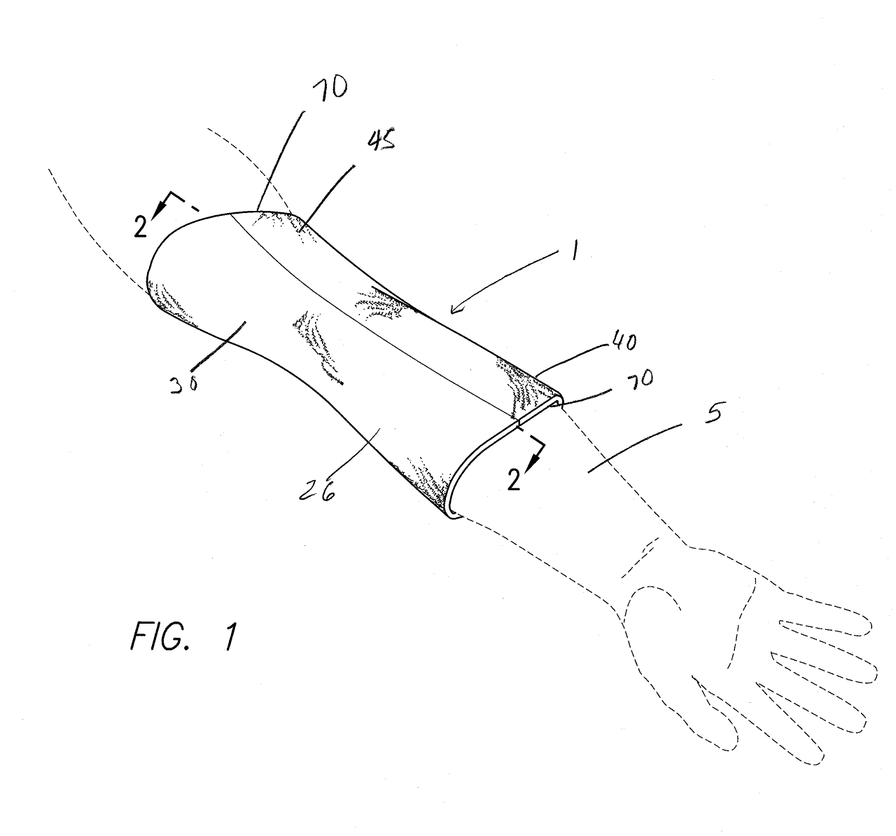

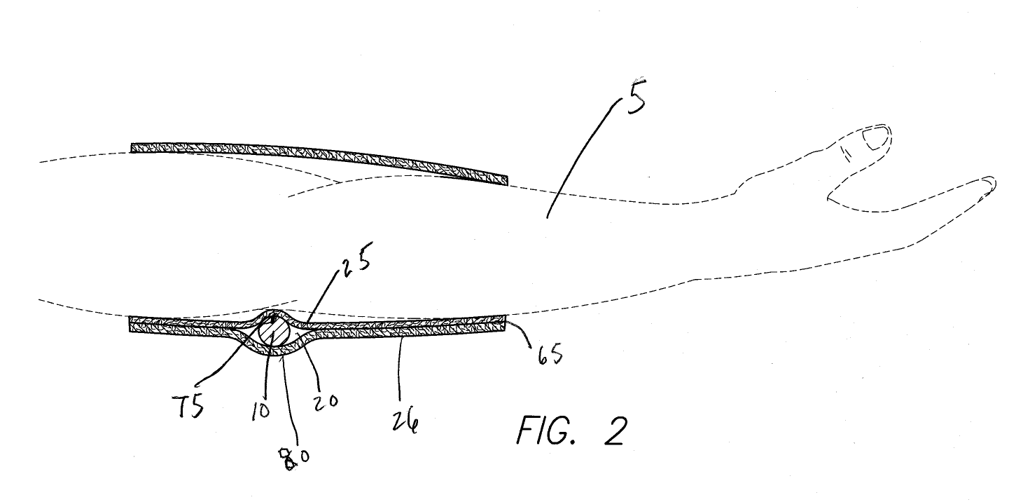

The lateral or circumferential elasticity of the sleeve allows it to expand and contract as the user's elbow flexes during normal movement of the body. Thus, a person wearing the device around his or her elbow as shown in FIGS. 1 and 2 is not impeded by the device from otherwise using the elbow.

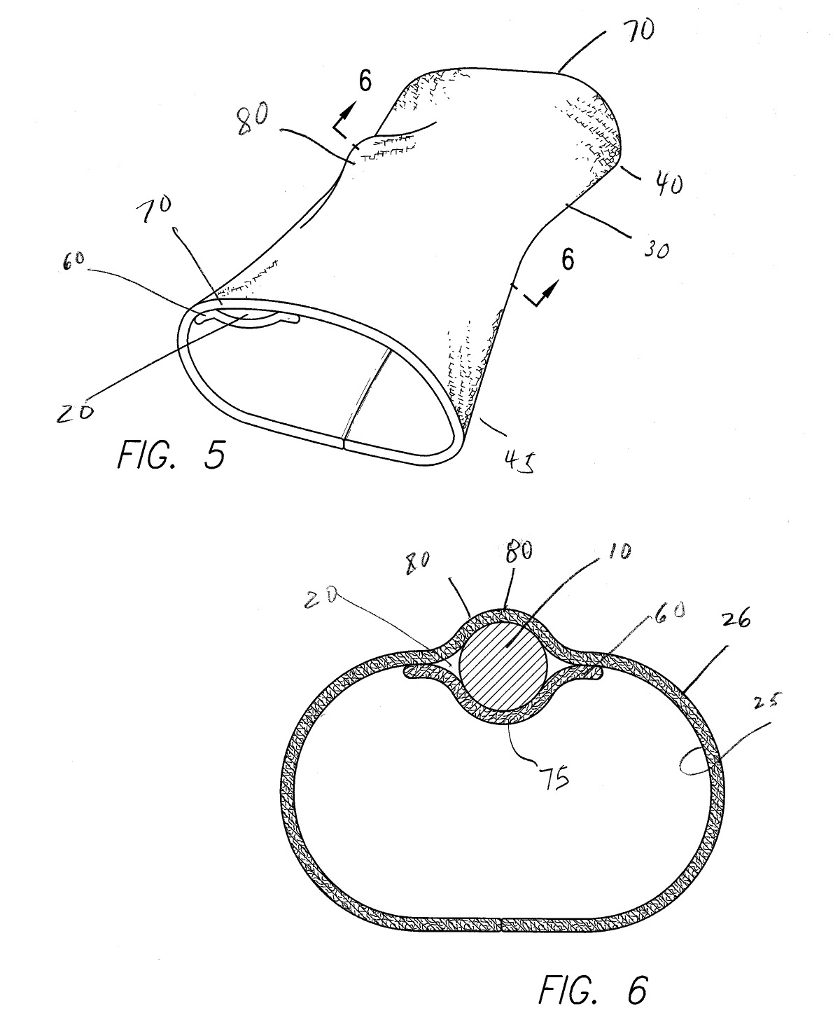

FIG. 2-6 show the sleeve 30 constructed in accordance with the present invention as positioned on the wearer's arm in the elbow region indicated generally at 55 in FIG. 1.

The invention comprises a tubular sleeve 30 preferably formed from any one or combination of one-way (circumferential or longitudinal) stretch fabric, two way stretch material, or non-stretchable material.

Sleeve 30 embodiments include, but are not restricted to longitudinal dimension ranging from about 4 inches to as long as about 10 inches and tapers slightly from a larger upper end 45 to a smaller lower end 40. It will, of course, be appreciated that the sleeve can be made shorter or larger if desired. In this manner, the sleeve conforms substantially to the shape of a wearer's arm in the elbow region. Methods and materials for making form fitting limb encircling therapeutic devices are well known in the art, for example, U.S. Pat. Nos. 3,934,583; 5,918,602, incorporated herein by reference.

### Material

Any well known type of material may be used such as a fabric-faced neoprene, and the like and this material may be a stretchable or non-stretchable. Cotton, everyday textiles, e.g. nylon polyester and or other synthetic fabrics compatible, i.e. wearable with the skin may be used for the fabric face of the neoprene. Non-limiting embodiments of such materials are set forth in Liangbing Hu, et. al., Nano Lett. 10, 708-714 (2010).

The sleeve of the invention is preferably fabricated from a woven or non-woven material, a surface of which is juxtaposed to the user's body and is relatively soft and comfortable in contact with a person's body. The material also should be sufficiently lightweight and porous to allow the material to “breathe” while wrapped around the body, so as to minimize sweating due to entrapment of perspiration. Preferred embodiments of the sleeve wrap comprise one or more materials selected from the group consisting of mesh, foam, elastic, and neoprene. The inner surface comprises a soft, pliable, wearable material, the material is selected from the group consisting of nylon, rayon and dacron.

Whatever the choice of material, it is important that the sleeve be relatively nonelastic in the longitudinal direction so that the selected overall length of the sleeve remains substantially unchanged as the device undergoes tension. The fabric of the device should be relatively elastic in the circumferential dimension, however, allowing the sleeve to undergo a substantial amount of circumferential stretching.

### Pocket

The sleeve is provided with one or more pockets 20 preferably formed on the interior or inner surface 25 of the sleeve 30. A pocket 20 accommodates and fits a removable pressure wad 10 or focal pressure applicator (FPA), at least a portion of its shape configured for focal pressure on a point of the user's elbow, and approximately spherical, semi-spherical, and in any case curved and formed from resilient or non-resilient material.

In accordance with the present invention, the pocket 20—, in which an FPA is snugly fitted—is disposed on the inner surface 25 of a circumferentially elastic sleeve so that when worn on the user's arm the elasticity causes a spherical surface of the FPA 10 to apply pressure on the desired point of the user's elbow, thereby relieving the elbow pain. The FPA 10 may be formed from any material which is substantially non-compressible, e.g. firm or hard rubber, approximately spherical, semi-spherical, and in any case curved and formed from non-resilient or substantially non-resilient material. Non-resilient materials suitable for forming an FPA10 include metal, plastic, wood, conglomerate or stone.

In certain embodiments, a pocket 20 comprises a sheet or tube of material fixedly positioned 55 on an inner surface 25 of the sleeve. Stitching is but one means for securing or forming a pocket. Other methods of securing or bonding sheets of materials to each other are well known in the art, and include, without limitation, application of adhesives or glue to the respective sheets of material. Successful long term bonding of elastomeric materials to themselves—and to other substrates—requires careful preparation and matching of the adhesive being used with the differing coefficient-of-expansion (COE) and chemistries of the substrates involved. (Reltek LLC, Santa Rosa, Calif.) The sheet 60 and the inner surface 25 of the sleeve 30 define a pocket 20 there between having an opening 65 at, respectively, one or both ends 40, 45 of the sleeve along one or both edges 70 of the sleeve

As shown in FIGS. 2, 4, 5 and 6 a pocket 20 formed by a 60 sheet and an inner surface 25 of the sleeve 30 accommodates an FPA 10 in snug fitting.

A pocket 20 is defined by securement or bonding positioned at 55 as seen in FIGS. 3, 4, 5 and 6.

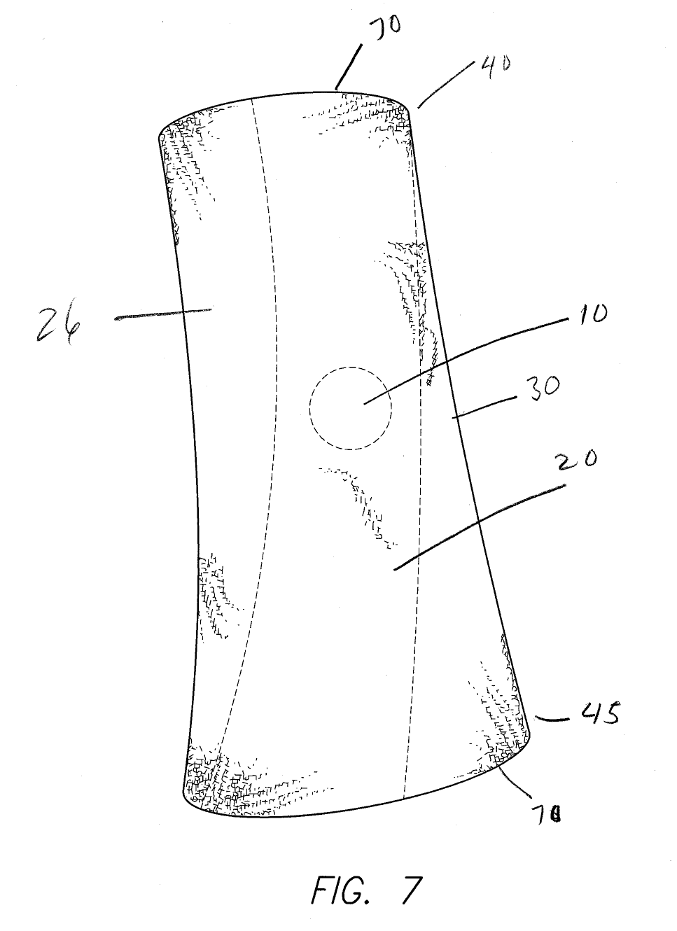

FIG. 7 presents an embodiment in which an elongate pocket is approximately the length of the sleeve. An embodiment of the FPA 10 is a sphere with a radius of approximately a half inch to about 1 inch (FIGS. 2, 3, 4, 6).

A pocket 20 is formed of material, not necessarily the same material as the remainder of the device, yet, as described above, wearable against the user's skin. The elongated embodiment of a pocket has a size defined in part by the size of the FPA intended to be inserted into the pocket. Pocket 20 is formed by a sheet of fabric integral with or secured to an inner surface of the sleeve and is provided with a relatively narrow opening 65 which can be stretched to permit the insertion and removal of an FPA 10. The pocket 20 is sized so that the FPA can be snugly inserted and snugly fitted anywhere along the length of the pocket so as to be in register with the desired position on the user's elbow.

Either end of the pocket 20 may be closed. A closed-end pocket is formed by stitching of the respective end of the pocket to the respective edge of the device.

### The FPA

The FPA 10 is positioned directly overlying or registered with a desired point on the elbow or forearm. The FPA defines a shape with at least a partially spherical surface whereby a point of the spherical surface exerts focal pressure on the user's elbow at an anatomical position desired by the user, generally where the application of focal pressure by the device results in the user experiencing pain relief.

The FPA 10 is shaped or it includes at least one surface which is spherical, semispherical or otherwise sufficiently curved with a radius having a point such that the device tensioned by an elastic or semi-elastic sleeve about the user's arm applies through the focal point of said FPA a therapeutic pressure (a focal therapeutic pressure) on a point on the user's elbow. In use, the sleeve is effective to apply a relatively high pressure on a selected point of the elbow vis a vis a point on a conical or spherical surface of the FPA, and providing optimum relief for the user's particular elbow condition. In accordance with the present invention, the FPA can be positioned so as to selectively provide a relatively high pressure directed to a relatively small area. Inserted and fitting snugly in a pocket, the spherical or conical point of pressure exerted by the operation of the device through the FPA is effective to apply a concentrated pressure against a relatively small surface of the elbow. Many wearers find that this highly concentrated pressure affords them optimum relief.

In the foregoing description, it was assumed the user had positioned the FPA in the pocket prior to placing the device on his or her arm. It should be apparent, however, that an FPA may be inserted before or after placement of the sleeve on the user's elbow.

An advantage of the device is that it can be positioned by the wearer upon the wearer's arm in different locations so that pressure can be concentrated on that portion of the wearer's arm which affords him the greatest relief. Accordingly, some wearer's obtain optimum relief by wearing the sleeve so that the FPA is positioned laterally in the region of the radial head. When the FPA is worn medially, i.e., on top of the wearer's arm so that pressure is applied to the brachioradialis muscle, other users obtain optimum relief. Other users, however, find that their condition is helped most effectively by periodically rotating the sleeve to a lateral position in which the FPA is disposed on the outside or inside of the arm in the region of the radial head. Other users may obtain benefit by raising the sleeve to a position with the FPA above the elbow.

One of the advantages of the present sleeve, which combines both a sleeve and a pocket positioned on an inner surface of the sleeve, is that once positioned on the arm, the sleeve is sized for snugly fitting the user's arm, thus the sleeve and the position of the FPA on a region of the elbow preferred by the user can be maintained in that position without the use of excessive tension or extraneous straps.

### Method of Treating Painful Conditions—Use of the Device

The present invention comprises a method for treating and alleviating stress and painful conditions of the myofascial structure, for example “tennis elbow”.

The sleeve of the present invention can be worn in a variety of positions to provide the type of support which benefits each particular wearer. In one step of the method, this position is determined empirically by the wearer.

The device is fitted snugly on the user's arm, in particular the elbow region and elbow-contiguous upper and lower portions of the arm. The device maintains or is retained in position in use irrespective of the flexed condition of the elbow, the device conforming to the elbow convexity as the elbow flexes. The size or number of pockets 20 positioned on the sleeve is considered to be matters of choice, depending primarily on the particular elbow application for which the wrap is intended.

It should now be understood that the present invention includes devices having sleeves of various lengths and circumferences which fit specific sizes of arms and elbows in which one or more FPA's are placed in pressured register with desired portions of the user's elbow. The device maintains or is retained in position in use irrespective of the flexed condition of the elbow, the device conforming to the elbow convexity as the elbow extends, flexes, and/or rotates. The user wears the device for a sufficient period to relieve pain in the elbow, or other joint around which the user applies an appropriately sized device to alleviate pain.

In any position, the elastic material of the sleeve, the pocket, and the non-elastic or relatively inelastic FPA are effective to grip the wearer's arm and function in combination to stably position the FPA in any desired position.

### The Wrap Embodiment

An embodiment of the device comprises a planar wrap which is furnished to the user with fastening devices or material (e.g. Velcro) affixed to free ends of the wrap. A sleeve embodiment is formed by means of at least one strip of fastening material affixed at one edge of the planar material and by at least one other strip 30 of complementary fastening material attached to the other side of the planar material. The complementary fastening strips may be provided by a hook-and-loop material such as Velcro material or other reversibly fastening, adhesive materials which include adhesive coating of a wide range of materials including: felt, foam, fabric, rubber, and film; laminating and laminating materials by which direct coating pressure sensitive and heat-activated adhesives are applied to a variety of soft or flexible materials. One of skill in the art has available well known companies (e.g. Associated Industries, Inc., Wichita, KS) which provide a variety of technologies and materials for securing soft materials to each other.

It should be apparent that the number of strips of fastening material used at the lateral edges of the planar material, particularly the number of longitudinally-aligned strips disposed in lateral spaced-apart relation adjacent lateral edges of the planar material depend on the physical dimensions of the planar material and of the strips of fastening material.

Accordingly, another embodiment of the device is an enclosing wrap of a predetermined configuration having an inner surface and an outer surface, free ends, and sized to enclose at least portions of the user's arm above and below the elbow. Fasteners (e.g. hook and loop type material) or other reversible securements affixed on free ends of the wrap permit reversible closure of the wrap around at least portions of the user's arm above and below the elbow when the free ends are overlapped.

The wrap embodiment has at least one pocket formed on an inner surface of the wrap. At least one pressure wad or FPA 10 comprising a rounded surface is disposed in said one or more pockets. In use, the user wraps the wrap around the elbow region, registers the fasteners or securements in overlapping configuration into engagement with each other thereby forming a sleeve snugly secured around the elbow region. The FPA 10 is adjustably registered by the user in therapeutic compression against a point on said user's elbow.

Preferred embodiments of the wrap comprise material selected from the group consisting of mesh, foam, elastic, and neoprene. The inner surface comprises a soft pliable, wearable material, the material is selected from the group consisting of nylon, rayon and Dacron.

Those skilled in the art will recognize that other forms of securement may be utilized in lieu of the Velcro tape. It will be appreciated that the lengths and widths of Velcro tape are sufficiently sized so that the user can fasten them to achieve varying degrees of tautness for accommodating arms of different sizes.

From the above disclosure of the general principles of the present invention and the preceding detailed description of a preferred embodiment, those skilled in the art will comprehend various modifications to which the invention is susceptible. Accordingly, the invention is limited only by the scope of the following claims.

1. A device comprising
(a) a sleeve sized for snugly encircling an arm in the region of the elbow;
(b) a pocket disposed on a length of a surface of said sleeve, said pocket sized for receiving a pressure wad,
wherein adjustment of either the position of said sleeve and/or the position of said pressure wad in said pocket registers a rounded surface of said pressure wad with an anatomical point on the elbow.

2. The device of claim 1 wherein at least one end of said pocket comprises an opening sized for receiving said pressure wad.

3. The device of claim 1 wherein at least a portion of the surface of said pressure wad is radially defined.

4. The device of claim 1 wherein the length of said sleeve is from about 4 inches to about 10 inches.

5. The device of claim 1 wherein said sleeve is formed from one or more materials selected from the group consisting of one-way stretch material, two-way stretch material, and non-stretchable material.

6. The device of claim 1 comprising a material selected from the group consisting of mesh, foam, elastic, and neoprene.

7. The device of claim 1 wherein the inner surface of said sleeve comprises a soft, pliable material.

8. The device of claim 1 wherein the material is selected from the group consisting of nylon, rayon and Dacron.

9. The device of claim 1 comprising a plurality of pressure wads disposed in one or more pockets.

10. The device of claim 1 comprising a plurality of pockets wherein one or more pressure wads is disposed in each of said pockets.

11. The device of claim 1 wherein said elongate pocket is on the outer surface of said sleeve.

12. The device of claim 1 wherein said elongate pocket is on the inner surface of said sleeve.

13. The device of claim 1 wherein a pocket is positioned on each of the inner and outer surfaces of said sleeve.

14. A device comprising:
(a) a wrap having an inner surface and an outer surface, free ends, and sized to enclose at least a portion of the user's arm above and below the elbow;
(b) releasable securements affixed on free ends of said wrap to permit the closure of the wrap around at least portions of the user's arm above and below the elbow;
(c) at least one pocket formed on the inner surface of the wrap embodiment of the device; and
(d) at least one pressure wad comprising a rounded surface disposed in said one or more pockets
wherein a user wraps said wrap around the elbow region, registers said securements in into engagement with each other thereby forming a sleeve secured around the elbow region, and wherein said pressure wad is registered in therapeutic compression against a point on said user's elbow.

15. The device of claim 14 wherein said wrap comprises a material selected from the group consisting of mesh, foam, elastic, and neoprene.

16. The device of claim 14 wherein said inner surface comprises a soft pliable material.

17. The device of claim 14 wherein the material is selected from the group consisting of nylon, rayon and Dacron.

18. The device of claim 14 wherein the securements comprise hook tape and loop.

19. A method for treating painful conditions of the myofascial structure of a joint, comprising the steps of:
(a) Applying the device of claim 1 to a joint; and
(b) Positioning said device in registration with the joint for applying focal pressure to said joint.
